(19) United States
(12) Patent Application Publication
(10) Pub. No.: US 2023/0059698 A1
(43) Pub. Date: Feb. 23, 2023

(54) Method of Manufacture for Liquid Cannabinoids, and Modified CBD Oil Composition

(71) Applicant: Honeysuckle Scientific, LLC, Flowood, MS (US)

(72) Inventor: Thomas L. Lucky, Canton, MS (US)

(21) Appl. No.: 17/845,499

(22) Filed: Jun. 21, 2022

Related U.S. Application Data
(60) Provisional application No. 63/235,391, filed on Aug. 20, 2021.

(51) Int. Cl. A61K 41/00; A61K 31/05; A61K 31/352

(52) U.S. Cl. CPC A61K 41/0004; A61K 31/05; A61K 31/352

(57) ABSTRACT

A method of manufacturing a cannabinoid solution. The method first comprises providing a solution containing a volume of cannabidiol (CBD), as an oil. Preferably, the solution comprises at least 80% by volume of the CBD oil. The method also includes placing the volume of CBD oil into a container. The method further comprises determining a vibrational frequency of energy. The method then includes applying the determined vibrational frequency of energy to the solution within the container in order to imprint the volume of cannabinoid solution with the determined vibrational frequency. A cannabinoid solution comprising a volume of cannabidiol (CBD), as an oil, is also provided. The solution has been imprinted with one or more selected vibrational frequencies of energy.

### STATEMENT OF RELATED APPLICATIONS

This application claims the benefit of U.S. Ser. No. 63/235,391 entitled “Method of Manufacture for Liquid Cannabinoids, and Modified CBD Oil Composition.” That application was filed on Aug. 31, 2021, and is incorporated herein in its entirety by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### BACKGROUND OF THE INVENTION

This section is intended to introduce selected aspects of the art, which may be associated with various embodiments of the present disclosure. This discussion is believed to assist in providing a framework to facilitate a better understanding of particular aspects of the present disclosure. Accordingly, it should be understood that this section should be read in this light, and not necessarily as admissions of prior art.

### Field of the Invention

The present disclosure relates to the field of cannabinoids. More specifically, the present invention relates to liquids containing cannabidiol (herein referred to as CBD oil) that have been infused with beneficial energy. The present invention further relates to a method of manufacturing a liquid cannabinoid solution.

### Discussion of Technology

Cannabinoids are naturally occurring compounds found in Cannabis sativa plants. Known cannabinoids include, for example, cannabidiol (CBD); cannabigerol (CBG); cannabichromene (CBC); tetrahydrocannabidivarin (THCV); cannabichromevarin (CBCV), tetrahydrocannabinolic acid (THCA); cannabidivarin (CBDV); cannabinol (CBN); and cannabidiolic acid (CBDA).

The most well-known cannabinoid is tetrahydrocannabinol (sometimes referred to as delta-9-tetrahydrocannabinol). Tetrahydrocannabinol is often abbreviated as “THC.” The chemical formula for THC is C₂₁H₃₀O₂ and it has the following chemical structure:

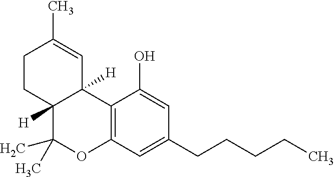 [SMILES: CCCCCc1cc(O)c2c(c1)OC(C)(C)[C@@H]1CCC(C)=C[C@@H]21]

The cannabis sativa plant produces hundreds of cannabinoids, terpenoids and other compounds. Cannabinoids activate cannabinoid receptors and ion channels in cells. The two main cannabinoids found naturally in the Cannabis sativa plant are THC and CBD. Typically, CBD represents up to 40% by weight in the extracts of the Cannabis sativa plant.

THC has recognizable psychoactive effects, meaning it makes a person ‘high’. In contrast, CBD is thought to have an anti-psychoactive effect that controls or moderates the ‘high’ caused by the THC. CBD is also thought to have various medicinal effects including, for example, pain management, help with sleep apnea, and reducing anxiety.

Different strains of Cannabis sativa plants exhibit varying levels of detectable CBD and THC as active cannabinoids. Therefore, an individual may select certain strains of plant based on its distinct cannabinoid levels, with the varying levels being either preferred or disfavored by the individual for a given purpose.

In recent years, companies such as United Cannabis Corporation of Golden, Colorado, have been formed to develop methods for extracting cannabinoids from Cannabis plants. U.S. Pat. Nos. 9,730,911 and 10,555,928 and have been issued to United Cannabis Corporation. These patents offer early methods of formulating pharmaceutically active cannabinoid compounds. Separately, patents have been issued to GW Pharma Limited describing cannabinoid-containing plant extracts for the treatment of epilepsy and neurodegenerative diseases. U.S. Patent Publ. No. 2021/0236458 discloses methods of pain management comprising the administration of specific formulations of cannabinoids.

Cannabinoids are sometimes placed into consumables such as an oil extract. For retail purposes, such oil extracts will contain a THC content of less than 0.3% of overall mass. In some instances, such as to improve taste, the oil extract will be blended with a coconut oil, a jojoba oil, an avocado oil, or other oil. An example of such an infusion is described in U.S. Patent Publ. No. 2021/0092972. Botanical ingredients such as peppermint, chamomile, basil, rosemary, ginger, eucalyptus, or citrus may also be added.

A need exists for a liquid formulation of cannabinoids that has been infused with specific frequencies in order to enhance the efficacy of CBD oil for medicinal or therapeutic benefits. A need further exists for a method of manufacturing liquid cannabinoids wherein a formulation containing CBD oil is infused with designated frequencies to enhance the phyto-therapeutic efficacy of the cannabinoid solution.

### SUMMARY OF THE INVENTION

A method of manufacturing a cannabinoid solution is first provided. The method first comprises providing a volume of a cannabinoid solution. The solution will contain cannabidiol (CBD), wherein the CBD is an oil. The CBD has preferably been extracted from the flowers, buds, or both, of hemp plants.

Preferably, the cannabinoid solution comprises at least 50% by volume CBD oil. Ideally, the cannabinoid solution comprises less than 0.3 percent by volume tetrahydrocannabinol (THC), and more preferably less than 0.1 percent. It is understood that THC is an aromatic terpenoid and is recognized as the principal psychoactive constituent in cannabis. More specifically, THC is the component responsible for the intoxicating effect in cannabinoid products.

The method also includes placing the volume of cannabinoid solution into a container. Ideally, the container is a glass container configured to transmit energy waves from outside the container and into the CBD oil residing within the container. In one aspect, the container holding the cannabinoid solution is sealed. In another aspect, the container is ultraviolet (UV) light-transparent.

In one embodiment of the method, the solution is cleansed of any pre-existing vibrations. Cleansing the solution of pre-existing vibration(s) may include using various techniques to disorganize and eradicate any pre-existing vibrations in the solution. This may be done by applying vibrations from one of a large variety of instruments. The varying vibrations are configured or selected to cancel out pre-existing vibrations in the solution so that few, if any, predominant vibrations exist in the solution after a cleansing. Alternatively, this may involve contacting the glass container with a cellulosic substrate, e.g., cloth, which may be used to dampen pre-existing frequencies. Simultaneously, the solution may be cooled to reduce energy.

The method further comprises determining a vibrational frequency of energy. More specifically, the method comprises selecting one or more vibrational frequencies of energy. The method then includes applying the one or more selected vibrational frequencies of energy to the volume of CBD oil within the container. This is done in order to imprint the cannabinoid solution with the determined vibrational frequency or frequencies. The determined (or selected) vibrational frequency or frequencies are imprinted into the cannabinoid solution to influence a desired effect of the solution upon an individual when ingested.

In one aspect, the one or more selected vibrational frequencies of energy corresponds to a determined frequency of a plant from the Lonicera genus.

In another aspect, the one or more selected vibrational frequencies of energy corresponds to a selected plant extract. These are referred to as essential oils. Preferably, the selected vibrational frequency resides between 48 and 550 MHz, inclusive.

The method may also include transferring the cannabinoid solution into individual UV light-reflective dropper bottles. This would be done for resale, distribution, and/or shipping purposes.

A cannabinoid solution is also provided herein. The cannabinoid solution comprises a volume of cannabidiol (CBD), as an oil. The cannabinoid solution has been imprinted with one or more selected vibrational frequencies of energy.

Preferably, the cannabinoid solution comprises at least 50% by volume CBD oil. The CBD has preferably been extracted from the flowers, buds, or both, of hemp plants and exists as an oil. Ideally, the cannabinoid solution comprises less than 0.3 percent tetrahydrocannabinol (THC).

The one or more selected vibrational frequencies of energy preferably resides between 48 and 550 MHz, inclusive. In one aspect, the one or more selected vibrational frequencies of energy corresponds to a determined energy of a plant from the Lonicera genus. Alternatively, the one or more selected vibrational frequencies of energy corresponds to one or more selected essential oils. In yet another arrangement, the one or more selected vibrational frequencies of energy corresponds to the measured frequency of a pharmaceutical product. The pharmaceutical product may be, for example, a drug used for the treatment of malaria or inflammation. Alternatively, the pharmaceutical product may be used for the treatment of Covid-19. Examples include remdesivir, chloroquine, hydroxychloroquine, or ivermectin.

The additional vibrational frequencies may also create a buffer in cannabinoid solution in order to increase and/or retain the infused vibrations in the solution for a sustained and predetermined period of time.

In one embodiment, the one or more selected vibrational frequencies of energy comprise:
a first vibrational frequency that corresponds to a first essential oil; and
a second vibrational frequency that corresponds to a second essential oil.

The first essential oil may be selected from the group comprising lavender, myrrh, sandalwood, peppermint, thieves, basil, melrose, frankincense, rose, Idaho blue spruce, angelica, juniper, helichrysum, and ravensara. Similarly, the second essential oil may also be selected from the group comprising lavender, myrrh, sandalwood, peppermint, thieves, basil, melrose, frankincense, rose, Idaho blue spruce, angelica, juniper, helichrysum and ravensara.

### BRIEF DESCRIPTION OF THE DRAWINGS

FIG. 1 is a flow chart showing steps for a method of manufacturing a cannabinoid solution of the present invention, in one embodiment.

### DETAILED DESCRIPTION OF CERTAIN EMBODIMENTS

### Description of Specific Embodiments

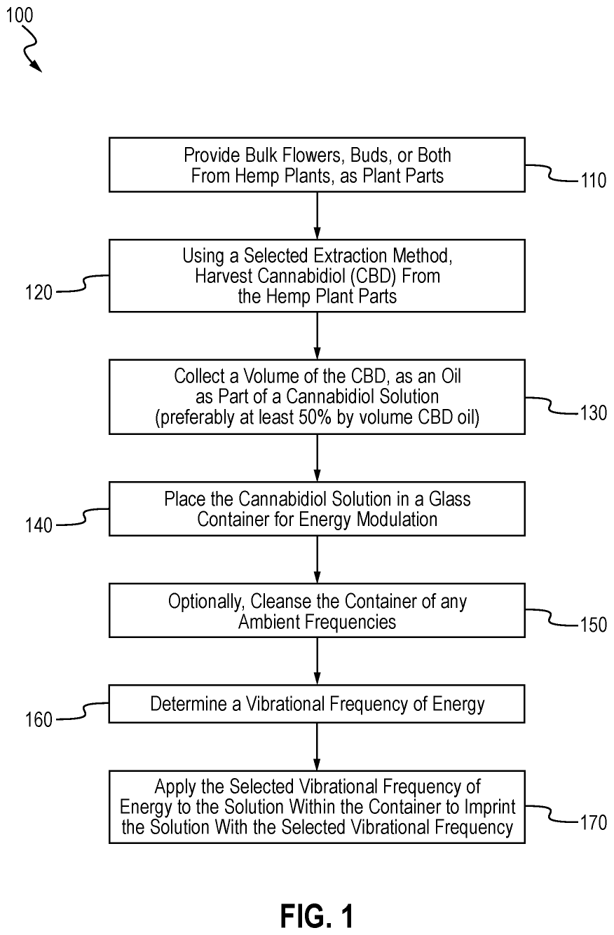

A method of manufacturing a cannabinoid solution is first provided. FIG. 1 is a flow chart showing steps for a method 100 of manufacturing a cannabinoid solution of the present invention, in one embodiment.

The method 100 first comprises providing plant parts from one or more hemp plants. This is provided in Box 110. The plant parts will typically include flowers, buds, or both, provided in select quantities from farms or agricultural warehouses. The plant parts may be from a single selected species of cannabis sativa or from a blend of species from plants of the genus Cannabis, representing hemp plants.

The method 100 next includes extracting cannabidiol (CBD) from the hemp plant parts. This is shown in Box 120. An operator may use any method to obtain a desired extraction. Distillation processes are preferred.

One method commonly used for extracting CBD is through pressurized carbon dioxide (CO₂). A closed-loop extractor pressurizes a container of CO₂ such that the CO₂ stays in liquid form instead of returning to its ambient gaseous state. The liquid CO₂ is pumped into a container of the selected cannabis plant parts. Through this process, the CO₂ absorbs the oils, flavors and scents of the plant parts. Upon being pumped into another less pressurized chamber, the CO₂ returns to its ambient gaseous state, leaving behind concentrated cannabis oils as a distillate.

Another CBD extraction method uses a high-grade grain alcohol (or ethanol) as a solvent to separate the cannabinoids from the plant parts. This process may be carried out at a variety of temperatures and pressures and is considered time-efficient when compared to other processes. However, ethanol extraction is prone to reducing the cannabis plant waxes which are sometimes desired when using the CBD oil as a consumable additive.

Another method of extraction is through the use of absorbent liquid solvents, wherein the solvents are run through decarboxylated hemp plant parts. Decarboxylation is the process that activates certain desired compounds in cannabis plants. Drying and curing cannabis over time will cause a partial decarboxylation to occur. Cannabis more fully decarboxylates when exposed to a heat of approximately 220° F. for 30-45 minutes. Of course, a time for substantial decarboxylation depends on a moisture content of the cannabis, the heating source employed, and other factors. It is important that the heating source employed does not burn or damage the batch of hemp during a heating process. At any rate, once a batch of hemp is decarboxylated, the liquid solvents can be used to break down the decarboxylated hemp into its oils.

Other extraction processes in support of the step of Box 120 may be employed to produce hemp oil extract. This disclosure is not limited to the specific method employed in Box 120.

The method 100 next comprises collecting a volume of the CBD in a liquid (or oil) state. This is presented in Box 130. As part of the step of Box 130, additional purification may take place. For example, to further purify the CBD extraction process, methods such as winterization are used. After the CBD oil is extracted, it is combined with high-grade alcohol and stirred until properly mixed. The mixture is then placed in a freezer at a below-zero Fahrenheit temperature. The alcohol thins the CBD oil, allowing the desirable parts of the oil to go into solution with the alcohol. At the same time, the less desirable parts coagulate and freeze, allowing them to be filtered out along with the fats when the solution is removed from the below-zero temperatures and run through paper filters. Once filtered, the solution is heated to the alcohol's boiling point. The boiling point of the alcohol will be less than that of the CBD oil. The alcohol boils off, leaving the purified CBD oil behind as the distillate.

An alternative purification process is the so-called “short-path distillation process.” Since each CBD oil has its own unique boiling point, by slowly heating the oil, the less-desired parts in solution, i.e., terpenoids, flavonoids, and contaminates begin to boil off. The boiled-off vapors can be collected through the use of a vacuum chamber operating at a lower pressure than the heating vessel. These vapors will travel until they reach cooling coils where they can be condensed and collected. This process is continued until only pure, or substantially pure, CBD oil is left in the original heating vessel.

In any instance, the cannabinoid solution preferably comprises at least 50% by volume CBD oil, and more preferably at least 80%, and further still at least 98%. Ideally, the cannabinoid solution comprises less than 0.3 percent tetrahydrocannabinol (THC), and more preferably less than 0.1 percent, and more preferably still less than 0.05 percent.

The method 100 also includes placing the volume of CBD oil into the container. This is seen at Box 140. Ideally, the container is a glass container configured to transmit energy waves from outside of the container into the cannabinoid solution residing within the container. In one aspect, the container holding the cannabinoid solution is sealed. In another aspect, the container is UV light-transparent.

The method 100 additionally and optionally includes cleansing the container of any ambient frequencies. This is seen in Box 150. Cleansing pre-existing vibrations of the solution may involve applying vibrations from one of a variety of instruments. The varying vibrations are selected to cancel out pre-existing vibrations in the solution so that few, if any, predominant vibrations exist in the solution after cleansing.

Playing one or more vibration instruments in variations of sequences or frequencies can be used. The process may be done at different speeds, directions, and/or magnitudes for a predetermined period of time depending on solution size and severity of pre-existing vibrations. In some embodiments, moving a vibration or sound in a radial direction, e.g., clockwise or counter-clockwise, with respect to the container of the solution can draw predominate vibrations away or cancel them out.

Alternatively, the cleansing process may involve physically contacting the glass container with a cellulosic or synthetic substrate, e.g., cloth or pad, wherein these materials are used to dampen the frequencies. Simultaneously, the solution may be cooled to reduce energy.

The method 100 further comprises determining a new vibrational frequency of energy. More specifically, the method comprises selecting one or more vibrational frequencies of energy. This is provided at Box 160 of FIG. 1. The method 100 then includes applying the one or more selected vibrational frequencies of energy to the volume of CBD oil within the container. This step is indicated at Box 170 of FIG. 1. Applying the one or more selected vibrational frequencies of energy to the volume of CBD oil within the container is done in order to imprint the cannabinoid solution with the determined vibrational frequency or frequencies. An imprinted cannabinoid solution is desirable as imprinting may increase the phyto-therapeutic efficacy of the cannabinoid solution.

The appropriate vibrational frequencies for the step of Box 170 can be achieved through a variety of techniques, wherein the CBD solution is exposed to frequency fields. This process may be referred to as “imprinting.”

Applying a determined vibrational frequency of energy may be done, for example, by exposing the volume of CBD oil to one or more magnetic fields. This may involve the use of actual magnets or the use of coils receiving a current to produce a magnetic field.

Alternatively, infrared lasers may be used. In one method, excitation of the CBD oil within the container employs multiple lasers oriented into the container at different angles and having several different frequencies.

Alternatively, the volume of CBD oil may be exposed to one or more signal generators. A signal generator may be one or more mechanical signal generators. Examples include an Earth gong, a drum, a bowl, a tuning fork, or a piano string, all of which would need to be struck to generate the mechanical energy. More preferably, the signal generator is an electro-mechanical device that emits a so-called ohm signal at a designated frequency or in a series of different frequencies.

In one embodiment, vibration instruments can generate low tones and low volumes with long frequencies. Then, tones generated by the vibration instruments can build in momentum allowing the frequency, the volume, or the sound magnitude to increase in order to bring the vibration instruments to crest. A crest or apex of the frequency, volume, or sound magnitude may then be used to induce the desired vibrational frequency into the CBD oil.

One or more musical instruments may be selected. Sound recordings of musical instruments may be played in proximity to the container at a selected volume. Acoustic instruments, primarily stringed acoustic instruments, are preferred in this embodiment.

The process of applying frequencies may be taken over a period of, for example, 2 minutes to 30 minutes depending on the source and intensity of the instrument used to apply the vibrational energy. A resonance can be created by interacting with the instrument, thus allowing a fluid medium to be imprinted by the frequency or frequencies.

In another method, various scalar techniques can be used to apply vibrational frequencies. Scalar techniques are techniques that manipulate the Morphogenic fields that organize the bodies of animals and plants through vibration patterns. Frequencies may be applied through the use of acoustic or stringed instruments, keyboard instruments, tuning forks, tuned bowls, and the like.

Of interest, a method known as dichroic lens filtering can be used to manipulate the selected vibrational frequencies. This is done through a thin film filter which interferes with certain wavelengths of light. This makes some wavelengths of light travel a slightly longer path, some light wavelengths are reinforced by this delay, while others tend to be canceled, producing the colors seen.

A vibrational buffer can also be added to the solution in order to increase and/or retain the infused vibrations in the solution for a sustained and predetermined period of time. This is done through a vibration instrument with high frequency capabilities. These frequencies will act to protect or neutralize typical disruptive environmental frequencies for a set amount of time depending on the amount of higher-level frequency added. The set amount of time may be decreased by introducing a greater amount of higher-level frequency, with an inverse of this phenomenon also being true.

In one aspect, the one or more selected vibrational frequencies of energy corresponds to a determined frequency of a plant from the Lonicera genus. The understanding behind this vibrational energy is that everything in our world vibrates on or at a specific frequency, including, for example, honeysuckle plants. Every particle, sound, color, and micro-organism carries its own energy frequency. The specific frequency that is applied to individual molecules has been determined to cause the molecule to “mimic” the effect of a specific substance or to cause a cell to do so.

In another aspect, the one or more selected vibrational frequencies of energy corresponds to a selected essential oil. Preferably, the selected vibrational frequency resides between 48 and 550 MHz, inclusive. Examples of essential oil frequencies are:

Rose: 320 MHz;

Helichrysum: 181 MHz;

Frankincense: 147 MHz;

Ravensara: 134 MHz;

Lavender: 118 MHz;

Myrrh: 105 MHz;

Juniper: 98 MHz;

Sandalwood: 96 MHz;

Angelica: 85 MHz; and

Peppermint: 78 MHz.

In one embodiment, applying the vibrational frequency of energy to the volume of CBD oil comprises:
selecting a first essential oil;
applying a first vibrational frequency to the volume of CBD oil that matches the vibrational frequency of the first essential oil for a first period of time;
selecting a second essential oil; and
applying a second vibrational frequency to the volume of CBD oil that matches the vibrational frequency of the second essential oil for a second period of time.

The first essential oil may be selected from the group comprising lavender, myrrh, sandalwood, peppermint, thieves, basil, melrose, frankincense, rose, Idaho blue spruce, angelica, juniper, helichrysum, and ravensara. Similarly, the second essential oil may also be selected from the group comprising lavender, myrrh, sandalwood, peppermint, thieves, basil, melrose, frankincense, rose, Idaho blue spruce, angelica, juniper, helichrysum, and ravensara.

In another embodiment, applying the vibrational frequency of energy to the volume of CBD oil further comprises:
selecting a plant from the Lonicera genus; and
applying a vibrational frequency of energy to the CBD oil for a third period of time that corresponds to a determined frequency of the selected plant from the Lonicera genus.

In one arrangement, the one or more selected vibrational frequencies of energy corresponds to the measured frequency of a pharmaceutical product. The pharmaceutical product may be, for example, a drug used for the treatment of malaria, or a drug used for the treatment of Covid-19. Examples include remdesivir, chloroquine, hydroxychloroquine, or ivermectin. Alternatively, the vibrational frequencies may correspond to selected herbal supplements such as curcuma longa, zinc, echinacea, ginger, and garlic.

It is believed and understood that by ingesting, either dermally or internally, CBD oil that has been imprinted with a selected frequency, the consumer is able to absorb the frequency into their body directly without relying upon the digestive organs.

In still another embodiment, the method may further comprise:
scanning the bio-energetic state of an individual to determine the frequency of the individual, wherein determining a vibrational frequency of energy comprises matching the bio-frequency of the individual.

Matching may mean providing a vibrational frequency that compliments the user's actual bio-frequency.

Of interest, the frequency of the vibrations imprinted in the solution can also be measured before and after the imprinting process. In some embodiments, the imprinting process, as described above, can be repeated one or more times in order to attempt to reinforce the effect of the imprinting process. The imprinting process can also be repeated at a later time, such as after four weeks of shelf life, in order to re-infuse or increase the strength of the previously-imprinted vibrations of the solution, which may otherwise naturally dissipate over a period of time.

The method 100 may also include transferring the CBD oil into individual UV light-reflective bottles. This optional step is shown at Box 180. This would be done for storage, resale, distribution, and/or shipping purposes. In some instances, the individual UV light-reflective bottles may be of a dropper type.

In addition to the method described above in connection with FIG. 1, a cannabinoid solution is also provided herein. The cannabinoid solution comprises a volume of cannabidiol (CBD) as an oil. The cannabinoid solution has been imprinted with one or more selected vibrational frequencies of energy according to the method 100, as described above.

Preferably, the cannabinoid solution comprises at least 50% by volume CBD oil, and more preferably at least 80%, and further still at least 98%. The CBD has preferably been extracted from the flowers, buds, or both, of hemp plants, as an oil. Ideally, the cannabinoid solution comprises less than 0.3 percent tetrahydrocannabinol (THC), and more preferably less than 0.05 percent.

The one or more selected vibrational frequencies of energy preferably resides between 48 and 550 MHz, inclusive. In one aspect, the one or more selected vibrational frequencies of energy corresponds to a determined energy of a plant from the Lonicera genus. Alternatively, the one or more selected vibrational frequencies of energy corresponds to one or more selected essential oils.

In one embodiment, the one or more selected vibrational frequencies of energy comprise:
a first vibrational frequency that corresponds to a first essential oil; and
a second vibrational frequency that corresponds to a second essential oil.

The first essential oil may be selected from the group comprising lavender, myrrh, sandalwood, peppermint, thieves, basil, melrose, frankincense, rose, Idaho blue spruce, angelica, juniper, helichrysum, and ravensara. Similarly, the second essential oil may also be selected from the group comprising lavender, myrrh, sandalwood, peppermint, thieves, basil, melrose, frankincense, rose, Idaho blue spruce, angelica, juniper, helichrysum, and ravensara.

In another aspects, the one or more selected vibrational frequencies of energy corresponds to the frequency of an individual based upon their bio-energetic state.

As can be seen, a unique method of manufacture for cannabinoid solutions has been provided. The method includes the imprinting of selected frequencies into the solution. A modified CBD oil solution is also disclosed. Those of ordinary skill in the art will understand that the scope of the inventions are defined by the claims, included below, and are not necessarily limited to any particular embodiments described herein.

I claim:

1. A method of manufacturing a cannabinoid solution, comprising:
providing a solution containing compounds of cannabinoids, as an oil;
placing the solution into a container;
selecting a vibrational frequency of energy; and
applying the selected vibrational frequency of energy to the solution within the container in order to imprint the solution with the selected vibrational frequency.

2. The method of claim 1, wherein the solution containing compounds of cannabinoids comprises at least 50% by volume cannabidiol (CBD).

3. The method of claim 2, wherein:
the CBD has been extracted from flowers, buds, or both, of hemp plants; and
the solution containing compounds of cannabinoids comprises at least 80% by volume CBD.

4. The method of claim 3, wherein the solution containing compounds of cannabinoids comprises less than 0.3 percent tetrahydrocannabinol (THC).

5. The method of claim 4, further comprising:
transferring the solution containing compounds of cannabinoids into individual ultraviolet (UV) light-reflective dropper bottles.

6. The method of claim 4, wherein the selected vibrational frequency of energy corresponds to a determined frequency of a plant from the Lonicera genus.

7. The method of claim 4, wherein the selected vibrational frequency of energy corresponds to a determined frequency of a selected essential oil.

8. The method of claim 4, wherein the selected frequency resides between 48 and 550 MHz, inclusive.

9. The method of claim 8, wherein applying the vibrational frequency of energy to the solution containing compounds of cannabinoids comprises:
selecting a first essential oil;
applying a first vibrational frequency to the solution that corresponds with the vibrational frequency of the first essential oil for a first period of time;
selecting a second essential oil; and
applying a second vibrational frequency to the solution that corresponds with the vibrational frequency of the second essential oil for a second period of time.

10. The method of claim 9, wherein the first essential oil is selected from the group comprising lavender, myrrh, sandalwood, peppermint, thieves, basil, melrose, frankincense, rose, Idaho blue spruce, angelica, juniper, helichrysum, and ravensara.

11. The method of claim 4, wherein applying the vibrational frequency of energy to the solution containing compounds of cannabinoids further comprises:
selecting a plant from the Lonicera genus; and
applying a vibrational frequency of energy to the solution for a period of time that corresponds to a determined frequency of the selected plant from the Lonicera genus.

12. The method of claim 1, further comprising:
scanning the energetic state of an individual to determine the bio-frequency of the individual;
wherein determining a vibrational frequency of energy comprises matching the frequency of the individual.

13. The method of claim 1, wherein applying the selected vibrational frequency of energy is done by exposing the solution to (a) one or more magnetic fields; (b) one or more infrared lasers, or (c) one or more mechanical signal generators.

14. The method of claim 13, wherein the one or more mechanical signal generators comprises an Earth gong, a drum, a tuning fork, an acoustic stringed instrument, a keyboard instrument, or a bowl.

15. The method of claim 1, wherein the container is a glass container.

16. The method of claim 15, further comprising:
after applying the selected vibrational frequency of energy to the solution within the container, placing the container in storage; and
after the container has been storage for a period of time, re-applying the selected vibrational frequency of energy to the solution within the container.

17. The method of claim 1, further comprising:
before applying the determined vibrational frequency of energy to the solution, cleansing the solution of any ambient or prevailing frequencies.

18. The method of claim 17, wherein cleansing the solution comprises (a) applying a cloth or pad to the glass container for a period of time, or (b) applying counter-frequencies to cancel out the ambient or prevailing frequencies for a period of time.

19. A cannabinoid solution, comprising a volume of cannabidiol (CBD) as an oil, wherein the CBD oil has been imprinted with one or more selected vibrational frequencies of energy.

20. The cannabinoid solution of claim 19, wherein the cannabinoid solution comprises at least 50% by volume CBD oil.

21. The cannabinoid solution of claim 20, wherein:
the volume of CBD oil has been extracted from flowers, buds, or both, of hemp plants; and
the cannabinoid solution comprises at least 80% by volume CBD oil; and
the one or more selected vibrational frequencies of energy resides between 48 and 550 MHz, inclusive.

22. The cannabinoid solution of claim 20, wherein the one or more selected vibrational frequencies of energy corresponds to the frequency of an individual based upon their bio-energetic state.
